(19) United States
(12) Patent Application Publication
(10) Pub. No.: US 2026/0113332 A1
(43) Pub. Date: Apr. 23, 2026

(54) System, Method, and Apparatus for Concurrent Processing and Role-Adaptive Presentation of Multi-Channel Communications

(71) Applicant: 1988 Venture Partners LLC Series 6, Rolling Hills Estate, CA (US)

(72) Inventors: Miriam Mackovic, Rolling Hills Estates, CA (US); Dzenana Gackic, Redondo Beach, CA (US)

(21) Appl. No.: 19/423,925

(22) Filed: Dec. 17, 2025

Related U.S. Application Data
(63) Continuation-in-part of application No. 17/493,635, filed on Oct. 4, 2021, now abandoned.

(51) Int. Cl. H04L 9/40; G06Q 30/0203; G16H 10/60

(52) U.S. Cl. CPC H04L 63/105; G06Q 30/0203; G16H 10/60; H04L 63/08

(57) ABSTRACT

A system and method directed at multi-channel communications are provided which may be implemented with an application executed on a computing device. A processor comprising the computing device may be operative to register and assign roles to various system users and grant users access to a plurality of administration modules according to each user's role and in response to receiving a single set of user-authenticating credentials. The plurality of administration modules may comprise a combination of communication, billing, service, marketing and analytics, and task modules.

### CROSS-REFERENCE TO RELATED APPLICATIONS

This application is a continuation-in-part of U.S. patent application Ser. No. 17/493,635 filed on Oct. 4, 2021, the entire contents of which are hereby incorporated by reference.

### GOVERNMENT CONTRACT

Not applicable.

### STATEMENT RE. FEDERALLY SPONSORED RESEARCH/DEVELOPMENT

Not applicable.

### COPYRIGHT & TRADEMARK NOTICES

A portion of the disclosure of this patent document may contain material which is subject to copyright protection. This patent document may show and/or describe matter which is or may become trade dress of the owner. The copyright and trade dress owner has no objection to the facsimile reproduction by any one of the patent document or the patent disclosure, as it appears in the Patent and Trademark Office patent files or records, but otherwise reserves all copyrights and trade dress rights whatsoever.

### TECHNICAL FIELD

The disclosed subject matter relates generally to electronic systems for customer relations management and more particularly to a unified system configured to facilitate client-service provider communication and business administration via a plurality of role-defined modules accessible by way of single-sign on user authentication.

### BACKGROUND

It is often the case that a healthcare provider may need to contact and coordinate a number of service providers to carry out administrative tasks such as billing patients, scheduling and providing appointment reminders, and storing and providing access to medical records, in addition to conducting medical care itself. That is, a number of likely unrelated parties may be involved in each step of scheduling, providing, recording, and billing for medical care, and even generating patient clients. These parties may include, for example only and not limitation, insurance providers, billing agents, customer service agents, scheduling agents, lead generators, and even doctors or other medical providers such as nurse practitioners and physicians'assistants and pharmacists themselves.

The same is true in other industries as well. For instance, product and service providers in the spa and beauty, hospitality, pharmaceutical, automotive, and consumer goods and appliance industries, and even those in telemarketing, sales, and distribution for the same and countless other industries, must necessarily communicate amongst themselves, with customers, and with third parties in all facets of administering their particular business.

Of course, in an era where communication, scheduling, invoicing and bill pay, and record keeping can occur largely online, patients and medical service providers as well as those other clients, customers, and service providers and administrators across industries alike can access and utilize many of these services from an electronic device such as a personal computer, laptop, tablet, or smartphone. Unfortunately, these services are generally maintained independently from one another over a multitude of software platforms and electronic accounts, each accessible to a user by its own unique log-in schemes, passwords, websites, and even software application downloads. Thus, business administration, and healthcare administration in particular, can be overburdened with the inconvenience of having to recall and manage each and every log-in credential, website, and application designated to enable any of the traditionally disassociated aspects of business administration and medical care and administration noted above.

Some have proposed solutions to these problems. For instance, in the healthcare filed in particualr, U.S. Pat. Pub. No. 2016/0103963 by Varun Mishra discloses streamlining medical care by generating smart medical records associated with a patient-identifying card. Likewise, U.S. Pat. Pub. No. 2015/0356250 by Marc Polimeni teaches a system in which health care providers generate electronic medical records in a web-based database, where ownership and control of such records remains with a patient. These references, however, fail to adequately address electronic communication between the patient and their health care providers. To that end, U.S. Pat. No. 10,854,320 to Fuhrmann is directed to improving patient-doctor communications in its workflow system for use in a hospital setting by way of a computer-implemented email messaging method. However, this and the foregoing references further fail to address the difficulty of assessing and collecting bill payments from patients. Moreover, none of these references are directed to unified systems that enable not only patient communication with their healthcare providers, but also billing, telehealth, and other healthcare-related services, together.

Thus, there remains a need for facilitating and delivering various aspects of customer services by an electronic system.

### SUMMARY

The present disclosure is directed to a system configured to facilitate client or customer-provider, manager-subordinate, patient-healthcare provider communication, among others via a plurality of role-defined modules accessible by way of single sign-on user authentication.

Broadly, the system comprises at least one electronic device operative to identify the role of an authenticated user and generate and display aspects of one or more communication, billing, service, marketing and analytics, and task modules in accordance with the identified role. Though each module may be otherwise functionally independent from each other module in the system, it is contemplated that not only may a user of any role access all of the modules assigned to their role using a single set of identifying and authenticating credentials, but also, each module may be readily accessible to a duly identified and authenticated user from a dashboard predefined for such user's role.

For purposes of summarizing, certain aspects, advantages, and novel features have been described. It is to be understood that not all such advantages may be achieved in accordance with any one particular embodiment. Thus, the disclosed subject matter may be embodied or carried out in a manner that achieves or optimizes one advantage or group of advantages without achieving all advantages as may be taught or suggested.

In accordance with one embodiment, the system comprises a computing device, such as a desktop computer, laptop computer, tablet, or smartphone, operative to execute a machine-readable website or software application that ultimately provides selective access to a plurality of independent modules according to the identity of a user. More particularly, the computing device is operative to register and assign a role to a user, and, responsive to receiving a set of user-specific identifying credentials, generate and display a plurality of relevant administration modules. For the sake of brevity and example only, but not limitation, healthcare may be discussed herein as an exemplary industry that may benefit from the invention, however, it shall be understood that other industries are contemplated as well. For instance, the administration modules may be directed to any of the healthcare, pharmaceutical, spa and beauty, automotive, hospitality and consumer good and appliance industries. More generally, the modules may be directed to telemarketing, sales, distribution, and customer service.

Thus, in light of the foregoing, one embodiment of the plurality of generated and displayed modules may be healthcare administration modules. The modules may be selected from communication, billing, service, marketing and analytics, task modules. In the case of the exemplary healthcare industry, the service module may be a telehealth module in particular. In other words, the system may be operative to combine communications, billing, service such as telehealth, marketing and analytics, and task modules into a single computer-executed application which is accessible to its users via an authenticated credential verifying each user's identity. The exemplary service as telehealth module may be replaced with other modules that facilitate delivery of relevant services between customers or clients and their providers in other industries as desired or needed without departing from the invention but may be discussed herein from time to time as the “telehealth” module for the sake of clarity in the exemplary embodiments given. In any event, it is contemplated that this allows each user to log in to the system once and access otherwise unrelated modules without re-entering authentication factors.

The particular combination of modules accessible to the user will depend on each user's role. In the nonlimiting example of healthcare provided, it is contemplated that a user of the system may be any of a healthcare provider such as a hospital administrator, doctor, nurse, physician's or nurse's assistant; another healthcare administrator; a billing agent; a customer service agent; a lead generator; or even any combination or group of the same. Thus, the system may be configured to assign a role to each user defined by such use's job or profession in the healthcare field. In such cases, the system may be configured to display and grant a user access to one or more of the plurality of modules and/or features defining such modules in accordance with their job.

In this regard, the disclosed system does not merely display information, but dynamically processes, classifies, and routes communications and associated tasks using automated execution logic that operates continuously and in parallel across multiple communication channels.

It will be understood that the system may be configured to grant users access to all of the modules according to their role by way of a single set of authenticating credentials. Various potential authenticating credentials are known to those of ordinary skill in the art and may be used as desired without departing from the invention. For instance, the set of authenticating credentials may be a preassigned or personally selected user name and password. In some embodiments, the set of authenticating credentials may be an email address or preassigned identification number or code, such as a quick response code or a bar code. The authenticating credentials may even be biometric in nature as defined by, for example, a user's fingerprint, eye print, or face. Of course, this is a nonlimiting list offered by way of example only and not limitation. Still, by arranging elements of the invention in this manner, a user may avoid a need of remembering or securely storing a plurality of sets of authenticating credentials. Indeed, a user even avoids a need to reenter authentication factors and other credentials in order to access each of the system modules available to them.

In some embodiments, the system is further configured to automatically orchestrate and process communications received concurrently across a plurality of independent communication channels, including telephone calls, web form submissions, chat messages, text messages, email messages, and system-generated notifications. The system automatically normalizes each received communication, resolves a contact identity by interfacing with one or more external record systems, evaluates multiple operational and contextual conditions in real time, classifies the communication, and generates and assigns structured tasks without requiring human intervention. These operations are performed concurrently for multiple communications, enabling real-time processing and routing that is not practically achievable through manual workflows.

Although modules are accessible to users according to each user's assigned role, much of the data stored within the modules and accessible to each independent, authenticated user may be unique to such user. For instance, if the user's role is defined as “customer service agent,” any communications, metrics, and records associated with that particular user's activities within the system may pertain to that user alone. As another example, for a user whose role is defined by managing, overseeing or aiding others, such as a system “admin” user, the system may be operative to generate and display other users'activities within the system.

The identity of a user may be associated with a single individual service provider such as an individual doctor, esthetician, mechanic, designer, sales representative, or other service provider according to this disclosure. It is also contemplated, however, that the identity of a user may be associated with a particular device, location, or administrative body. As a clarifying example, a particular computing device within a medical practice or hospital may be identifiable by the system as a credentialed user. Likewise, all users assigned a particular role, such as all of an individual doctor's assistants may be identifiable by the system as a credentialed user. As such, the particular characteristics of each user, whether as individuals, as a predefined group, or else, will not limit the invention.

Once a user is registered and identified within the system by a computing device, such device generate, display, and receive a selection of any of the administration modules available to them. In response to receiving such selection, the computing device may be operative to generate and display a dashboard associated with each of the plurality of modules and reflecting the functionality of the same. Thus, in an embodiment, a communication dashboard may be generated and displayed, as well as or in addition to a billing, service, marketing and analytics, and task dashboard. In some embodiments, a “home” dashboard is generated and displays all modules pertaining to the user's role together. Exemplary embodiments of each module will be discussed in turn, below.

In accordance with one embodiment of the invention, the system may further comprise a database comprising a plurality of individualized service records. In the exemplary embodiment comprising healthcare administration modules, such records may comprise patient health records, also known to those having ordinary skill in the art as electronic medical records or “EMR”s, in addition to the foregoing aspects and features of the invention.

Certain additional features of the invention are contemplated. For instance, the system may be operative to receive biographical user information and personal details as may be required or desired. Additionally, the system may be operative to rearrange the appearance and other aspects of the various embodiments of the dashboard(s) as appearing on any user interface associated with the system according to the personal tastes and preferences of each identified user without departing from the invention.

In light of the foregoing, it is one object of the invention to improve and even streamline communications between clients and service providers, such as, for example only and not limitation, healthcare service providers including doctors and their administrative assistants.

It is another object of the invention to increase client engagement with service providers.

It is yet another object of the invention to improve office workflow.

It is an object of the invention to condense traditionally distinct modalities into a single electronic system in order to reduce the number of accounts, agents, managers and/or other points of contact typically involved in traditionally independent systems.

It is another object of the invention to reduce service costs such as medical service costs.

It is another object of the invention to reduce delays in pricing, approving, and delivering services, such as healthcare services.

It is still another object of the invention to support an ongoing and more transparent relationship between clients and service provider.

It is yet another object of the invention to improve client education and awareness of services offered by certain service providers such as medical service providers.

It is still another objection of the invention to concurrently receive and process communications across multiple independent channels in real time.

It is another objection of the invention to automatically resolve contact identities, retrieve associated communication history, and generate structured tasks based on evaluated conditions without requiring human intervention.

It is yet another object of the invention to improve system scalability, responsiveness, and operational consistency by performing rule-based communication classification and task orchestration in parallel for multiple communications.

One or more of the above-disclosed embodiments, in addition to certain alternatives, are provided in further detail below with reference to the attached figures. The disclosed subject matter is not, however, limited to any particular embodiment disclosed.

### BRIEF DESCRIPTION OF THE DRAWINGS

FIG. 1 is a block diagram of a networked environment in which an exemplary embodiment of a system for concurrent processing and role-adaptive presentation of multi-channel communications is implemented.

FIG. 2 is a flowchart depicting an exemplary embodiment of a method for concurrent processing and role-adaptive presentation of multi-channel communications directed at the generation of dashboards as a result of user authentication.

FIGS. 3A and 3B is a flowchart depicting an exemplary embodiment of a method for concurrent processing and role-adaptive presentation of multi-channel communications directed at live operations in multi-channel communications and task management.

FIG. 4 illustrates an exemplary embodiment of an electronic device shown in FIG. 1.

FIG. 5 is a block diagram depicting an exemplary embodiment of a system for concurrent processing and role-adaptive presentation of multi-channel communications.

FIGS. 6-10 illustrate exemplary user interfaces in accordance with some embodiments.

One embodiment of the invention is implemented as a program product for use with a computer system. The program(s) of the program product defines functions of the embodiments (including the methods described herein) and can be contained on a variety of computer-readable storage media. Illustrative computer-readable storage media include, but are not limited to: (i) non-writable storage media (e.g., read-only memory devices within a computer such as CD-ROM disks readable by a CD-ROM drive) on which information is permanently stored; (ii) writable storage media (e.g., floppy disks within a diskette drive or hard-disk drive) on which alterable information is stored. Such computer-readable storage media, when carrying computer-readable instructions that direct the functions of the present invention, are embodiments of the present invention. Other media include communications media through which information is conveyed to a computer, such as through a computer or telephone network, including wireless communications networks. The latter embodiment specifically includes transmitting information to/from the Internet and other networks. Such communications media, when carrying computer-readable instructions that direct the functions of the present invention, are embodiments of the present invention. Broadly, computer-readable storage media and communications media may be referred to herein as computer-readable media.

In general, the routines executed to implement the embodiments of the invention, may be part of an operating system or a specific application, component, program, module, object, or sequence of instructions. The computer program of the present invention typically is comprised of a multitude of instructions that will be translated by the native computer into a machine-readable format and hence executable instructions. Also, programs are comprised of variables and data structures that either reside locally to the program or are found in memory or on storage devices. In addition, various programs described hereinafter may be identified based upon the application for which they are implemented in a specific embodiment of the invention. However, it should be appreciated that any particular program nomenclature that follows is used merely for convenience, and thus the invention should not be limited to use solely in any specific application identified and/or implied by such nomenclature.

For simplicity and clarity of illustration, the drawing figures illustrate the general manner of construction, and descriptions and details of well-known features and techniques may be omitted to avoid unnecessarily obscuring the invention. Additionally, elements in the drawing figures are not necessarily drawn to scale. For example, the dimensions of some of the elements in the figures may be exaggerated relative to other elements to help improve understanding of embodiments of the present invention. The same reference numerals in different figures denote the same elements.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements, but may include other elements not expressly listed or inherent to such process, method, system, article, device, or apparatus

The terms “couple,” “coupled,” “couples,” “coupling,” and the like should be broadly understood and refer to connecting two or more elements or signals, electrically, mechanically or otherwise. Two or more electrical elements may be electrically coupled, but not mechanically or otherwise coupled; two or more mechanical elements may be mechanically coupled, but not electrically or otherwise coupled; two or more electrical elements may be mechanically coupled, but not electrically or otherwise coupled. Coupling (whether mechanical, electrical, or otherwise) may be for any length of time, e.g., permanent or semi-permanent or only for an instant.

The present invention may comprise some elements of traditional database encryption, storage, management, and access. However, the present invention may also make use of other, more widely-used technologies, such as cloud computing, emails, and text messages. The present invention may also draw upon, digitize, or otherwise incorporate paper documents in some embodiments.

### DETAILED DESCRIPTION

Having summarized various aspects of the present disclosure, reference will now be made in detail to that which is illustrated in the drawings. While the disclosure will be described in connection with these drawings, there is no intent to limit it to the embodiment or embodiments disclosed herein. Rather, the intent is to cover all alternatives, modifications and equivalents included within the spirit and scope of the disclosure as defined by the appended claims.

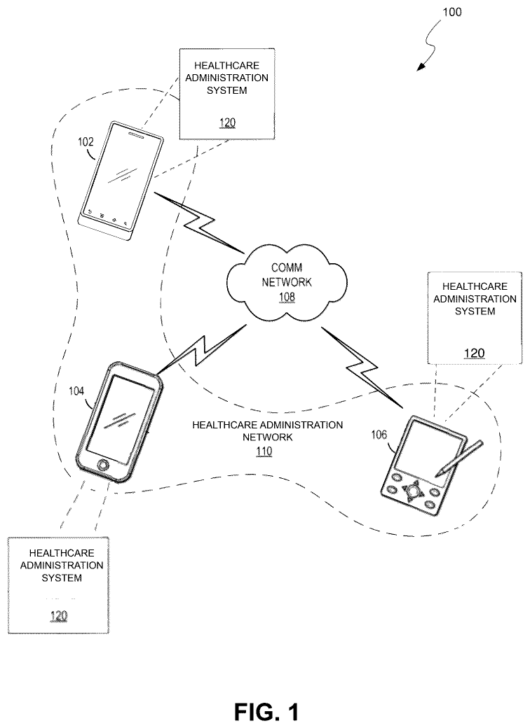

A description of an embodiment of a method and system for concurrent processing and role-adaptive presentation of multi-channel communications amongst members in a networked environment is now described followed by a discussion of the operation of various components within the system. In this regard, FIG. 1 is a block diagram of a networked environment in which an exemplary embodiment of a system for concurrent processing and role-adaptive presentation of multi-channel communications is implemented.

FIG. 1 illustrates an exemplary embodiment of the system for concurrent processing and role-adaptive presentation of multi-channel communications as a system for healthcare administration 100 which includes a plurality of computing devices, here depicted for example only as mobile devices or tablets and smartphones, communicatively coupled to one another. By way of example, and not limitation, FIG. 1 illustrates three mobile devices 102, 104 and 106 communicatively coupled via a communication network 108 which may generally be known to those of ordinary skill in the art. Each of the mobile devices may be embodied as a mobile computing device such as, for example and without limitation, a smartphone or tablet computer that incorporates cellular telephone functionality. Notably, the communications network can use one or more of various communications types such as, for example and without limitation, cellular and Wi-Fi communications.

Users of mobile devices 102, 104 and 106 may create an administration network such as the exemplary healthcare administration network 110 centered around a particular medical practice, group, practitioner, care facility, hospital, or other healthcare provider. These are known as the network center.

The healthcare administration network 110 enables members to use their devices 102, 104 and 106 to access modules defining the system and conduct various aspects of health care. Typically, a communication network is facilitated by a website that may require a registration and login prior to use. However, regardless of how such a communications network is implemented (be it web-based or not), the functionality of concern involves the ability to assign a role to a user and enable interaction with a plurality of role-specific healthcare administration modules.

In one embodiment, one way to add a user 102 to the communication network to send an invitation to another user's device 104 inviting them to join the network 110. Sending a network invitation to another member will prompt them to enter identification information such as, by way of example, name and email address. Upon complying with the membership requirements, the new member will be given access, using their mobile device 104, to all of the privileges enjoyed by the first member on their device 102.

In operation, the system 100 provides a better and more coordinated way in which various service providers, such as healthcare providers, interact with one another and their clients, such as patients. This includes doctors and their medical and administrative assistants, billing services providers, insurers, marketing and analytics providers, and any other individual or service provider that may be involved in running or providing support to a medical care provider. Specifically, the system comprises a computing device, such as a desktop computer, laptop computer, tablet, or smartphone, operative to execute a machine-readable application that ultimately provides selective access to a plurality of independent modules according to the role corresponding to the identity of the user. These modules may be selected from any combination of communication, service, billing, marketing and analytics, and task modules, though it is contemplated that the computing device will provide each user access to a role-specific communication module at the very least.

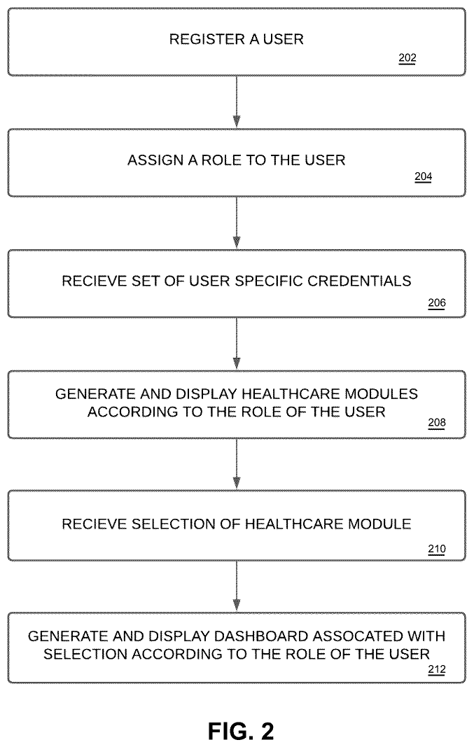

FIG. 2 is a flowchart depicting an exemplary embodiment of a method for concurrent processing and role-adaptive presentation of multi-channel communications such as may be performed by the mobile device 102 104, and 106 of FIG. 1 or another computing device. As shown in FIG. 1, the computing device is operative to register a user (block 202); assign a role to a user (block 204) responsive to receiving a set of user-specific, identifying credentials (block 206); generate and display a plurality of healthcare administration modules (block 208). These may be selected from communication, billing, service such as telehealth, marketing and analytics, and task modules, though it is contemplated that each user will have access to the communications module at the very least. Then, responsive to receiving a selection of a healthcare administration module (block 210) corresponding to the user's role, generate and display a dashboard associated with such selection, again according to the role of the user (block 212). Each of the exemplary healthcare administration modules, or “modules” in short, will be discussed in turn for the sake of providing clarifying examples only and not limitation.

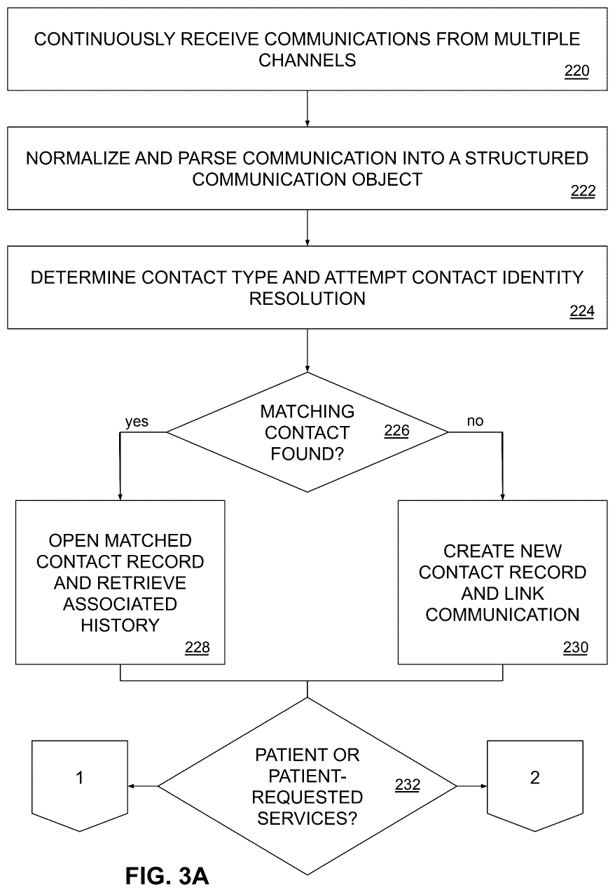

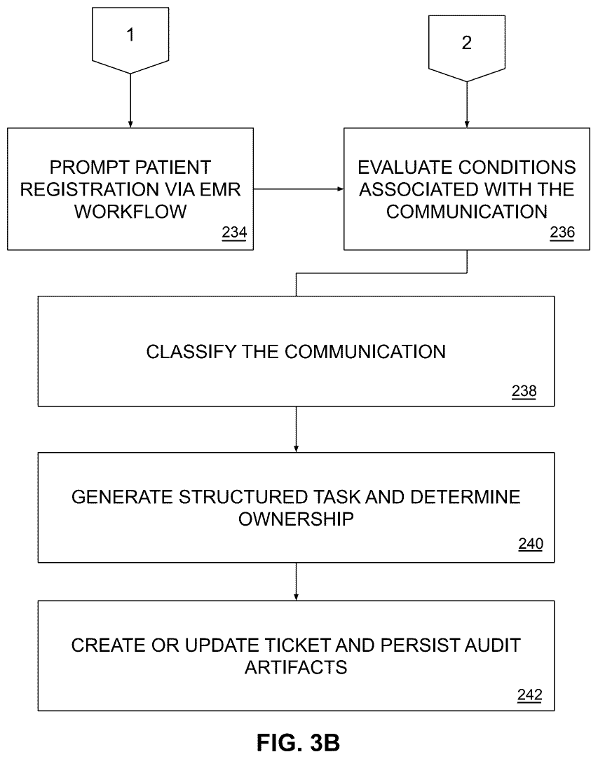

FIGS. 3A and 3B are flowcharts depicting an exemplary embodiment of an automated, computer-implemented workflow for orchestrating multi-channel communications and task management, such as may be performed by the computing devices and systems described herein after a user has been registered, authenticated, and granted role-based access to one or more administration modules as described with respect to FIG. 2. In this embodiment, the system operates continuously and in real time to receive, evaluate, classify, and route incoming communications, even simultaneously incoming communications, across multiple independent channels, while avoiding reliance on human intervention.

As shown in FIG. 3, the system is configured to continuously receive communications and asynchronous inputs from a plurality of independent communication channels (block 220). Such channels may include, by way of example and not limitation, telephone calls, web form submissions, chat messages, text messages (SMS), email messages, and system-generated notifications. Each communication may originate from or be directed to a different external entity, such as, for example only and not limitation, hospitals, pharmacies, patients, laboratories, etc., and may occur independently of other communications, such that multiple communications may be received and processed concurrently across different channels.

Responsive to receiving a communication, the system is operative to normalize and parse the communication into a structured communication object (block 222). In this step, the computing device may be configured to extract and standardize channel-specific metadata and content to generate a unified record usable across channels, including, for example, sender and destination identifiers; timestamps; channel identifiers, message subject and content payload, and any associated attachments or metadata. By normalizing communications in this manner, the system enables uniform downstream processing regardless of the originating channel.

The system may then automatically determine a contact type associated with the communication and attempts contact identity resolution (block 224). In particular, the system evaluates whether the communication corresponds to a non-patient contact type stored within the system, such as a referring provider, pharmacy, vendor, laboratory, or other external stakeholder, or a patient or a request for patient-related services corresponding to a patient demographic record stored in an external electronic medical records (EMR) system. This determination is performed automatically by the computing device without requiring user input.

If a matching contact record is identified (block 226), the system is operative to open the matched contact record and retrieve associated historical data (block 228), including prior communications, prior tickets or tasks, and other interaction history linked to the contact. If no matching contact record is found, the system automatically creates a new contact record within the system and associates the communication with the newly created record (block 230).

If the communication is determined to be associated with a patient or a request for patient-related services (block 232), the system initiates or prompts a patient registration workflow through an external EMR system (block 234). In this regard, patient demographic records remain stored within the EMR, while the system maintains associations to communications, tasks, and operational artifacts without storing patient demographic data locally.

Following contact resolution, the system automatically evaluates a plurality of conditions associated with the communication (block 236). Such conditions may include, by way of example and not limitation, the communication channel, the resolved contact type, message content or extracted keywords, time of day and office hours, provider or staff availability, and predefined operational and/or clinical rules. This evaluation may be performed in real time by the computing device.

Based on the combined evaluation of the communication content and associated conditions, the system is configured to automatically classify the communication into a structured category or type (block 238). Exemplary classifications may include scheduling requests, prescription refill requests, laboratory result inquiries, referral coordination, administrative requests, or other predefined categories.

Responsive to classifying the communication, the system automatically generates or updates a structured task or ticket and determines ownership of the task (block 240). Ownership may be assigned to an individual system user or a team of users based on one or more of role-based routing rules, evaluated conditions, current queue state, and availability information. This task generation and assignment occurs automatically, and avoids manual intervention by human users. Indeed, it is crucial to note that execution of the foregoing task generation and assignment logic would be impractical, if not impossible, to perform manually, as human operators would be required to simultaneously monitor multiple independent communication channels, identify and resolve contact identities, retrieve and interpret prior communication history, evaluate operational and availability conditions, and apply routing rules in real time for each incoming communication. Moreover, because communications may be received concurrently across different channels and may involve different contact types and conditions, manual execution would be unable to consistently perform such evaluations with the accuracy, timeliness, and concurrency required. By contrast, the system performs these operations automatically and in parallel using a computerized execution environment, thereby enabling real-time task orchestration and routing that cannot be feasibly achieved through human workflows alone. As a result, the system improves computational efficiency, scalability, and response latency by enabling concurrent, rule-based task orchestration across multiple communication channels in a manner that reduces processing delays, minimizes routing errors, and maintains consistent operational behavior as communication volume and contact diversity increase.

The system is further configured to route notifications to the determined assignee(s) and update one or more interface displays. Such updates may include, for example, updating task queues or ticketing panels, updating contact timelines, and updating dashboards associated with the assigned role. Notifications may be transmitted via one or more communication channels supported by the system.

The system may then record the communication as a ticket and persists resulting actions in an auditable log or communication transcript (block 242). Such records may include linkage to the associated contact record, task assignments, timestamps, and actions taken in response to the communication.

While the foregoing steps are being performed for a given communication, the system continues to receive and process additional communications across other channels concurrently. As such, identity resolution, condition evaluation, classification, task generation, routing, and logging may be performed in parallel for multiple communications in real time, enabling scalable operation that is not practically achievable through manual workflows. In some embodiments, the foregoing steps are performed concurrently for multiple communications received across different channels. Additionally, the external EMR system may be a separate system boundary. That is, patient demographic records are stored in the EMR while the system itself stores non-patient contact records and communication-related artifacts.

In accordance with one embodiment, a communication module may be configured to facilitate communication between a user of the system and client, such as any exemplary patients discussed herein. In some embodiments, each module may be defined as a modular microservice.

In an embodiment, the system is operative to generate and display a single or home dashboard in which all possible modules are viewable to and accessible to users of the system. In some embodiments, however, the system may be operative to generate and display a particular combination of modules to users defined by a particular role. The particular combination of modules generated and displayed for each user may be selected based on its functionality and depending on the users role. That is, the system may be operative to display an “agent,” “administrator,” “patient,” “client” “manager,” “doctor” or other dashboards that generate and display modules comprising relevant functionality depending on the performance requirements of individuals in such role.

Aspects of a dashboard corresponding to the communication module, of the “communication dashboard,” of the exemplary embodiment, may itself be generated and displayed according to the role of the user. For instance, roles may be administrators, managers, agents, doctors, or others, and the particular functionality and/or display generated by the computing device may reflect a workflow associated with such role. The particular workflow contemplated is boundless, but for the sake of example, may include corresponding with patients or clients, coworkers, and other service providers via chat, email, phone, and/or paper. The system may be configured to generate and display reports and performance information and even recorded communications generated and displayed on behalf users assigned a junior role to users assigned supervisory or managerial roles within the system. Thus, the system, enabled by one or more computing devices, may be operative to facilitate user management of and interaction with aspects of their own profile, view any leads and/or tickets associated with their role-specified modules, review any call and chat ratings as may be generated by interactions with features in such modules, and facilitate manager-user review of junior users.

To that end, the system may comprise an electronic device operative to facilitate communication over a plurality of channels. For instance, one or more communication channels may define a voice channel in which, for example only and not limitation, patients or other clients, depending on the industry, are enabled to provide personal information, facilitating personalized voice call-backs, or storing and transmitting voice recordings. As another example, communication channels may comprise, additionally or alternatively, a chat bot configured to communicate standardized information, or even for storing and/or transmitting chat transcripts by and between users. As still another example, communication channels may define web forms configured to receive patient, or client, and/or administrator selections that system records as a scheduled or cancelled appointment, as the case may be. Still other, or additional, aspects of the communication channels may define view lab results, refill prescriptions and others. An SMS communication channel may be provided to transmit and receive messages between users of the system, provide automated messages such as appointment reminders and prescription notices, as well as store message history. An email communication channel can further be provided to permit users to communicate with one another via email, store email history, and provide automated messages via email. More generally, in some embodiments, the communications module may comprise, an integrated customer support and patient or other client liaising communications channel.

A computing device comprising the system may be further configured to execute operations that improve workflow efficiency in the communication module. For instance, the communication module may comprise a Contact Control Panel (CCP), known to those skilled in the art, configured to receive and execute instructions to create and manage user or network contacts, who may be patients, clients, doctors, suppliers, and the like, and track automatic leads.

Certain other aspects of the system, enabled by the computing device, may be configured to, for example, push notifications to patients or other clients when predetermined events occur. For example, the system may be configured to generate and send to the patient or other client and relevant service provider a remote meeting or appointment link, or even an appointment reminder from within the communication module.

In one embodiment of the invention, a billing module provided and configured to verify insurance benefits and collect associated payments as well as bill and collect patient or other client payments. The system is thereby configured, by a computing device, to process and collect payments through multiple user interfaces and financial instruments. The computing device may be operative to calculate and assess insurance benefits, including copays and deductible amounts, in advance of medical service in order to reduce the traditional lack of transparency that patients and other clients and even medical and other service providers have come to expect in terms of payment.

In some embodiments, a payment schedule may be assigned, and periodic payment reminders may be automated and pushed through to various users.

Payment balances and records of each service provided may be viewed together. Data may be pushed and recorded in the aforementioned electronic record, such as an electronic medical record associated with each patient or client, after such patient or other client's payment is processed. In some embodiments, it is contemplated that this will minimize processing times, procedural approvals, and ultimate delivery of medical services. For instance, insurance benefits may be immediately viewed and accessed within the system, where traditionally, one would need to have the medical information forwarded to the insurance, billed separately, and then ultimately approved. Integrating a patient or other client billing process with the insurance providers in this manner thus also reduces overall billing time and optimizes communication between service providers, such as medical providers, and insurance companies and patients or other customers alike. It is further contemplated that client billing becomes more transparent and may be optimized through payment scheduling and automatic payment reminders enabled within the system.

In some embodiments, the system may further comprise a service module. In the exemplary embodiment, such module may be a telehealth module. That is, the system may be operative to facilitate electronic meetings between, for instance, health care providers and their patients such as through virtual telehealth appointments, again via the single-sign on system disclosed herein. Such a scheme may enable patients and other clients to check in electronically prior to an in-person office visit. Of course, more generally, the system may be operative to facilitate electronic meetings between any service provider and client via the single sign-on system without departing from the invention.

Any intake documents or other forms associated with the performance or receipt of medical care or other service, as the case may be, may be delivered, filled out, and collected online via the telehealth module, or other relevant service module, prior to the appointment, and then automatically stored in the electronic records database. In some embodiments, such functionality may be executed in the communication module instead of the telehealth or other service module.

The system, by the aforementioned exemplary computing device, may be further operative to electronically receive, push to, and store any biographical client information, such as identification cards, credit cards, and insurance cards within the electronic record database.

Patients and other clients, depending on the industry, can view and pay any costs prior to or following appointments. As above, this functionality may be executed in the service module as a telehealth module or billing module. It is executed within this exemplary telehealth module by way of example only, and not limitation. In some embodiments, the system may be operative to receive amendments to billing totals. This will of course depend on the role of the user. For example, a user-customer service agent may not be empowered by the system to amend billing totals. However, such user may push any related requests to modify billing totals to an empowered user to address the same.

That is, the system is operative to remotely receive and store administrative filings, including documents, identifications, and others, and even calendar information, from patients or other clients, and service providers such as doctors directly in a secure electronic records database. Telehealth appointments may occur within the system so that a record of the appointment is automatically incorporated into the electronic medical records database.

In some embodiments, the system may comprise a marketing and analytics module configured to support various business aspects of providing medical care. For instance, the system, by a computing device, may be operative to automatically deliver any marketing campaigns carried out by a medical provider amongst a predetermined or selected demographic. Indeed, in some embodiments of the invention, the system may be operative to aggregate and sort patients and others in terms of role and even demographic information including, for instance, geographic location, age, and family status to ensure that any material dispersed to a plurality of patient or client-users including, marketing information, may be relevant to such users.

In some embodiments, predesigned templates may be selectable by users assigned certain roles—such as marketing coordinators—within the system for amendment and distribution within the system.

Likewise, the system may be operative to automatically push and receive responses to patient and/or other client surveys to streamline the solicitation of information that could improve the subject administration. Relatedly, the system may further be operative to collect email and click link percentages, comparing them over time such as month to month or over a period of time, and by certain demographic filters.

As noted above, the marketing and analytics module, as with any other module comprising the invention may comprise its own dashboard, and any features generated and/or displayed on such dashboard may be selected or provided based on the role assigned to the user of the system.

In addition to the communication module which may be accessible to every user of the system, a task module may be generated and displayed to each user of the system. Of course, the particular functionality accessible to the user will depend on the user's role. Still, it is contemplated that a task module may be configured to, in general, automate task management processes. That is, the system, by a computing device, may generate and display certain tasks automatically, depending on the role of the user. But also, each user may be empowered by the system to generate and prioritize their own tasks as well as any tasks of those users whose assigned role indicates that they manage or supervise. The system may further generate and display tickets, known to those of skill in the art, according to selected, assigned, or automated task priority. In some embodiments, task requests received by the system may be automatically delegated to users based on information characterizing a user's predefined expertise.

In some embodiments, the system may be configured to generate and display task reports may to provide visibility into a related group of users'performance and response time. This may be particularly useful for a group of customer service users, for instance, or even a group of doctors within a single hospital or care network. Such reports may include subjective patient or other client and peer or supervisor user reviews as well as objective measures such as timing, number of served individuals, and the like.

Enabled by the exemplary computing device, the system may further comprise search functionality that allows various users to search contacts, leads, workflow tickets and/or requests, and any communications messages occurring across the system. Further, in some embodiments such as the exemplary healthcare administration, the system may be operative to collect, store and access for patient or other client documents as a fully integrated electronic medical record. It is contemplated though that in some embodiments, electronic medical records may be stored and accessed from a database outside of but accessible to computing devices comprising the system. Either of these functionalities may be defined by an independent module or incorporated into existing modules such as the communication module, service modules, or exemplary telehealth modules. Of course, it is contemplated that such functionality will be generated and displayed for use by a user depending on that user's assigned role within the system.

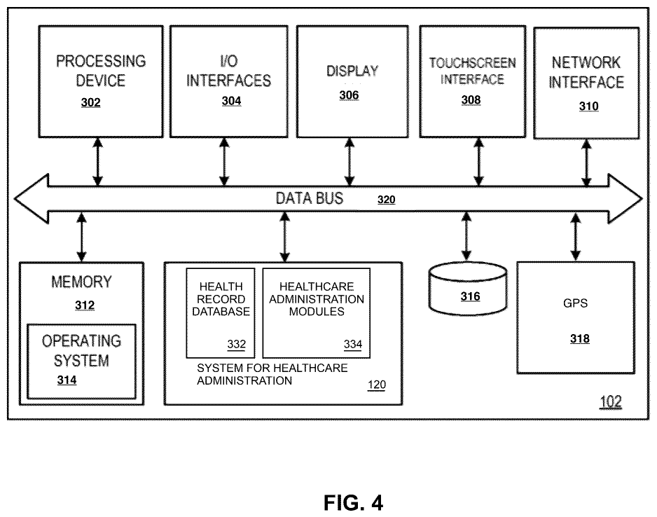

FIG. 4 illustrates mobile device 102 shown in FIG. 1. As described earlier, the computing device 102 may be a laptop, desktop or tablet computer or smartphone but may also be embodied in any one of a wide variety of wired and/or wireless computing devices. As shown in FIG. 3, computing device 102 includes a processing device (processor) 302, input/output interfaces 304, a display 306, a touchscreen interface 308, a network interface 310, a memory 312, and operating system 314, a mass storage 316 and a GPS 318, with each communicating across a local data bus 320. Additionally, computing device 102 incorporates an exemplary system for concurrent processing and role-adaptive presentation of multi-channel communications as a system for administering healthcare 100, which is depicted as including a health record database 332, which may be an database of electronic medical records associated with all patients cared for by user medical providers, and health care administration modules 334 configured according to the information provided, although the location of information 332, 334 and 336 could vary.

The processing device 302 may include any custom made or commercially available processor, a central processing unit (CPU) or an auxiliary processor among several processors associated with the computing device 102, a semiconductor based microprocessor (in the form of a microchip), a macroprocessor, one or more application specific integrated circuits (ASICs), a plurality of suitably configured digital logic gates, and other electrical configurations comprising discrete elements both individually and in various combinations to coordinate the overall operation of the system.

The memory 312 can include any one of a combination of volatile memory elements (e.g., random-access memory (RAM, such as DRAM, and SRAM, etc.)) and nonvolatile memory elements. The memory typically comprises native operating system 314, one or more native applications, emulation systems, or emulated applications for any of a variety of operating systems and/or emulated hardware systems, emulated operating systems, etc. For example, the applications may include application specific software which may comprise some or all the components of the mobile device 102. In accordance with such embodiments, the components are stored in memory and executed by the processing device. Note that although depicted separately in FIG. 3, the exemplary system and method for healthcare administration 100 may be resident in memory such as memory 312.

Touchscreen interface 308 is configured to detect contact within the display area of the display 306 and provides such functionality as on-screen buttons, menus, keyboards, etc. that allows users to navigate user interfaces by touch. For some embodiments, the mobile device 102 will comprise GPS 318 or other means to determine the location of the mobile device 102.

One of ordinary skill in the art will appreciate that the memory 314 can, and typically will, comprise other components which have been omitted for purposes of brevity. Note that in the context of this disclosure, a non-transitory computer-readable medium stores one or more programs for use by or in connection with an instruction execution system, apparatus, or device. With further reference to FIG. 4, network interface device 310 comprises various components used to transmit and/or receive data over a networked environment such as depicted in FIG. 1. When such components are embodied as an application, the one or more components may be stored on a non-transitory computer-readable medium and executed by the processing device.

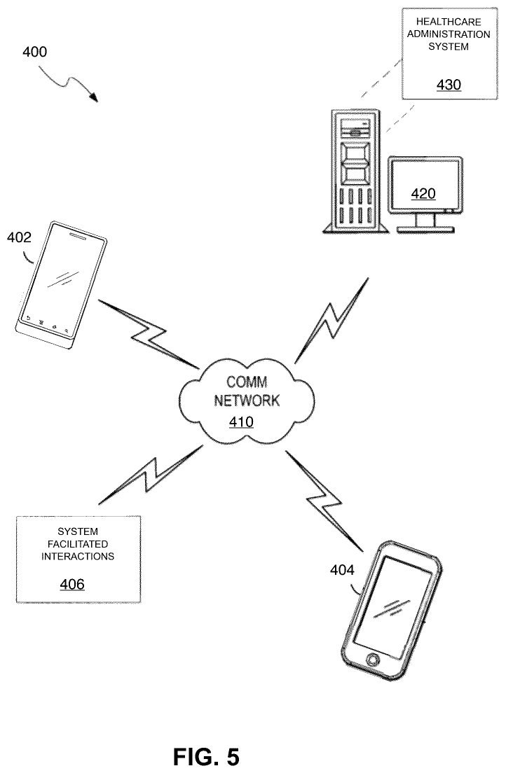

FIG. 5 is illustrative of an alternative networked environment in which another exemplary embodiment of a system for concurrent processing and role-adaptive presentation of multi-channel communications is implemented. As shown in FIG. 4, system 400 comprises a plurality of computing devices as mobile devices or smartphones. By way of example, and not limitation, two mobile devices 402 and 404 are shown communicatively coupled via a communication network 410. Each of the mobile devices may be embodied as a mobile computing device such as, for example and without limitation, a smartphone that incorporates cellular telephone functionality. Notably, the communications network can use one or more of various communications types such as, for example and without limitation, cellular and Wi-Fi communications.

Users of mobile devices 402 and 404 may use their devices to become members of a medical communications network that enables them to interact with each other using their mobile devices 402 and 404 and exchange communications as system facilitated interactions 406 according to their role and as allowed by modules comprising the exemplary healthcare administration system 430. In this exemplary embodiment, the medical communications network 410 is facilitated by a website or software application, as the case may be, that is hosted by the fully integrated healthcare systems server 420. For the purpose of the example presented in FIG. 5, the members of the network includes the users of mobile devices 402 and 404.

It is contemplated and worth noting that the foregoing is offered by way of example only, and not limitation. The medical communications network may be configured in a serverless and/or microservices computing architecture known to those having ordinary skilled in the art. In such embodiments, any modules accessible to each user, of course dependent on the user's role, may be decoupled from each other module in a manner that increases performance, reliability, and scalability. This is because any specific modules or functional applications within such systems are effectively distributed from one another and therefore maintain their independent from one another. It is in this manner that each user may be granted access to their own role specific modules and related dashboards. Users as subscribers to the network and specific modules perform different functions from one another and can therefore be enabled to perform different tasks or interact with data in each module in their own role-specific way.

In some embodiments, the system is distributed across a plurality of servers. Thus, FIG. 4 is offered as a clarifying example and for the sake of brevity.

In some embodiments, an exemplary server 420 implements the system for concurrent processing and role-adaptive presentation of multi-channel communications and facilitates sharing of communications and/or interactions between members of the medical communications network 410. Specifically, the server 420 implements the steps outlined in FIG. 2. Accordingly, member of the medical network can access the server 420 using their computing devices 402 and 404 to communication or otherwise interact, according to the modules accessible to them on account of their assigned role within the network,

In order to facilitate the aforementioned functionality, various aspects may be performed by one or more of the mobile devices 402, 404. In one embodiment, mobile device is operative to perform, at least in part, the method depicted in the flowchart of FIG. 2 as noted above. More particularly, the computing device may be operative to register and assign a role to a user, and, responsive to receiving a set of user-specific, identifying credentials, generate and display a plurality of healthcare administration modules selected from communication, billing, service such as telehealth, marketing and analytics, and task modules. Likewise, and with respect to operation of system for concurrent processing and role-adaptive presentation of multi-channel communications 430, system 430 may be operative to perform, at least in part, the method depicted in the flowchart of FIG. 2.

As embodied in software, it should be noted that each block depicted in the accompanying flowcharts represents a module, segment, or portion of code that comprises program instructions stored on a non-transitory computer readable medium to implement the specified logical function(s). In this regard, the program instructions may be embodied in the form of source code that comprises statements written in a programming language or machine code that comprises numerical instructions recognizable by a suitable execution system such as the mobile device 102, 104, 106, 402 and 404. The machine code may be converted from the source code, etc. If embodied in hardware, each block may represent a circuit or a number of interconnected circuits to implement the specified logical function(s). Additionally, although the flowcharts show specific orders of execution, it is to be understood that the orders of execution may differ.

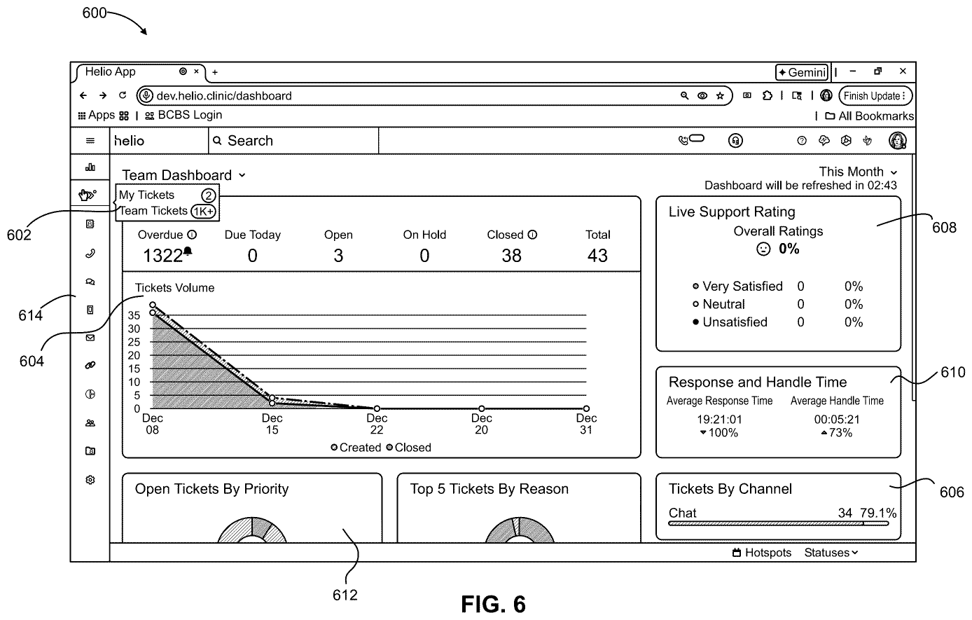

FIG. 6-10 illustrate exemplary graphical user interfaces generated and displayed by the system to support automated, real-time orchestration of multi-channel communications and task management as described herein. The depicted interfaces are provided by way of example only and are not intended to limit the scope of the invention.

FIG. 6 illustrates an exemplary team dashboard interface 600. In this embodiment, the dashboard displays user-specific tickets and team-level tickets 602 concurrently, along with dynamically updated operational statistics. Such statistics may include, for example, ticket volume over time 604, tickets categorized by communication channel 606, support ratings 608, response times and handling times 610, and open ticket priorities 612. The dashboard may be automatically updated in real time as communications are received, classified, and converted into tasks or tickets by the system, thereby providing a consolidated, role-adaptive view of ongoing system activity without requiring manual aggregation by human users. The dashboard further includes selectable interface elements 614 corresponding to one or more administration modules, which, when selected, cause the computing device to generate and display module-specific views and associated data according to the role of the authenticated user.

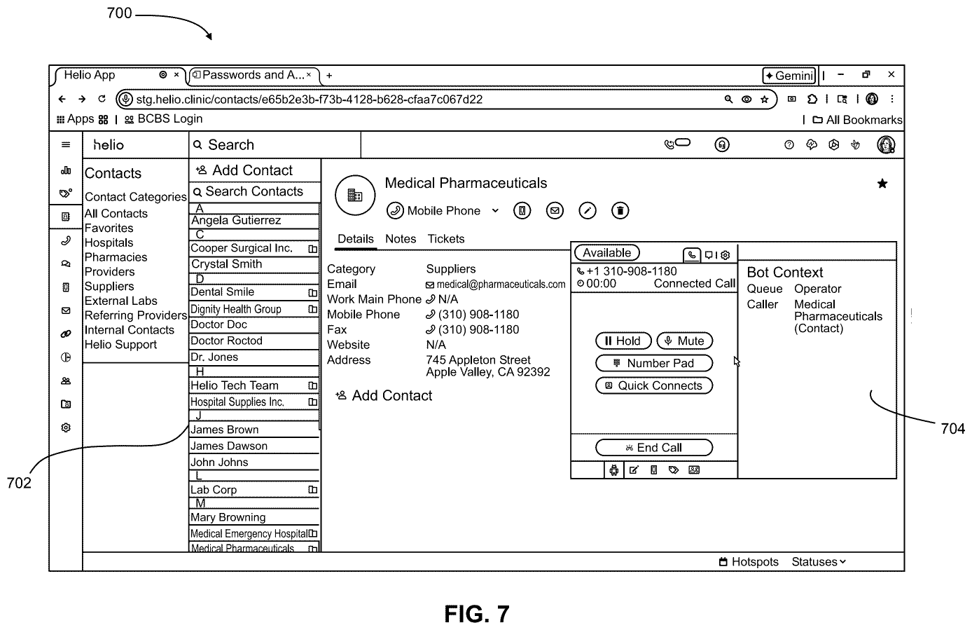

FIG. 7 illustrates an exemplary contacts interface 700. The contacts interface displays contact records 702 organized by contact category, which may include patients, providers, pharmacies, laboratories, vendors, or other external stakeholders. In the illustrated embodiment, the interface further includes a dynamically surfaced pop-up 704 corresponding to an incoming communication, such as a telephone call, associated with a known contact. The pop-up is generated automatically by the system upon resolving the contact identity and provides immediate contextual information to the user while the communication is being processed.

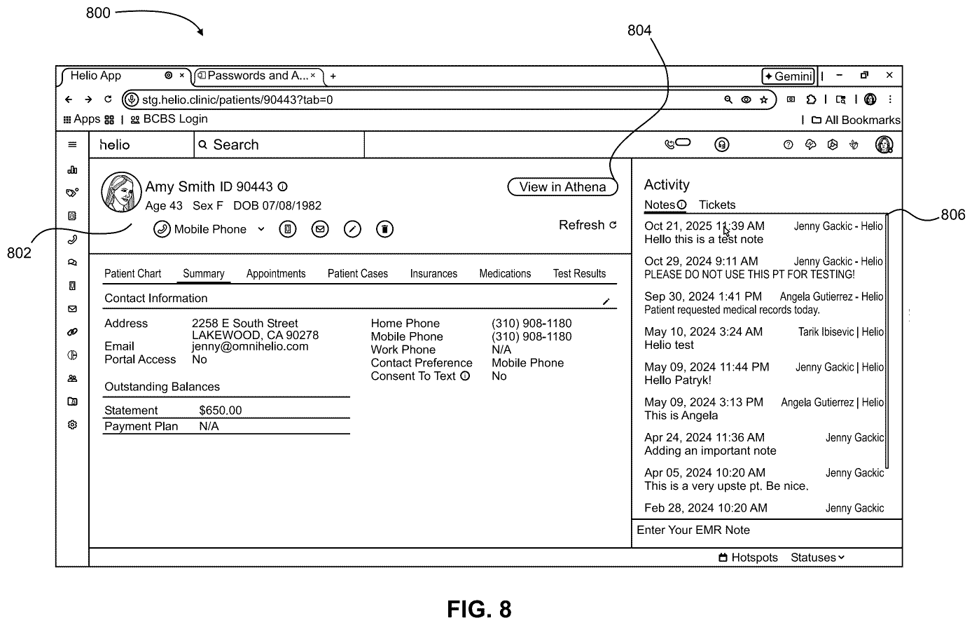

FIG. 8 illustrates an exemplary contact page 800 for a patient contact. The interface includes identifying information for the contact, a selectable control for navigating to an external electronic medical records (EMR) system 804, and a consolidated view of activity notes and tickets associated with the contact 806. In this manner, the system maintains communication artifacts, tasks, and operational records while patient demographic records remain stored in the external EMR system. The displayed activity and ticket information is automatically updated as communications are received and processed by the system.

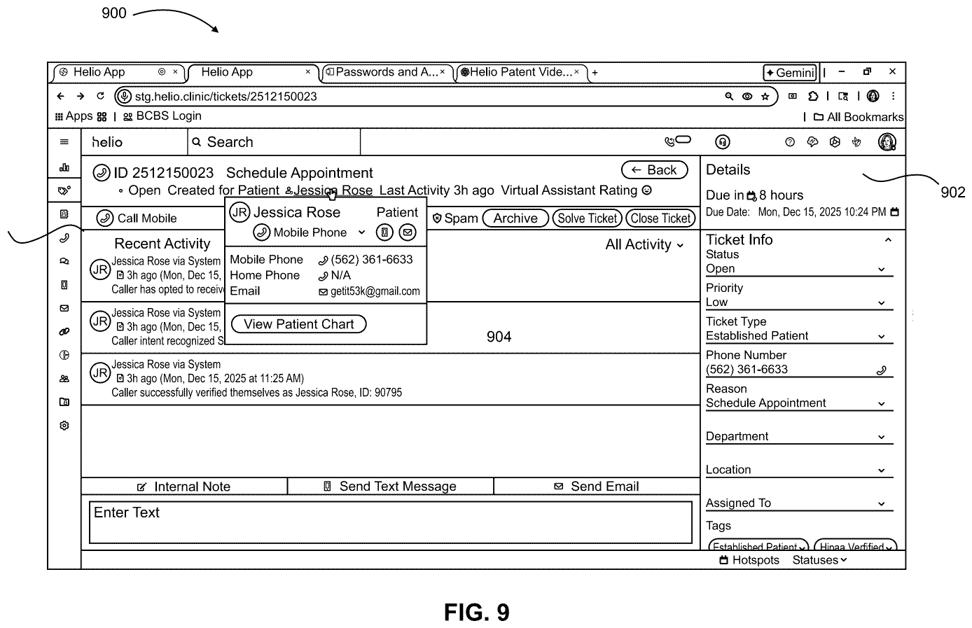

FIG. 9 illustrates an exemplary ticket interface corresponding to a task directed to scheduling an appointment 900. The interface displays ticket details 902 including status, priority, ticket type, and contact classification (e.g., established patient). The interface further displays system-generated activity entries 904 associated with the ticket, such as automatically recorded communications, routing actions, and task updates linked to the resolved contact. These activity entries are generated and saved automatically by the system as part of the real-time orchestration workflow.

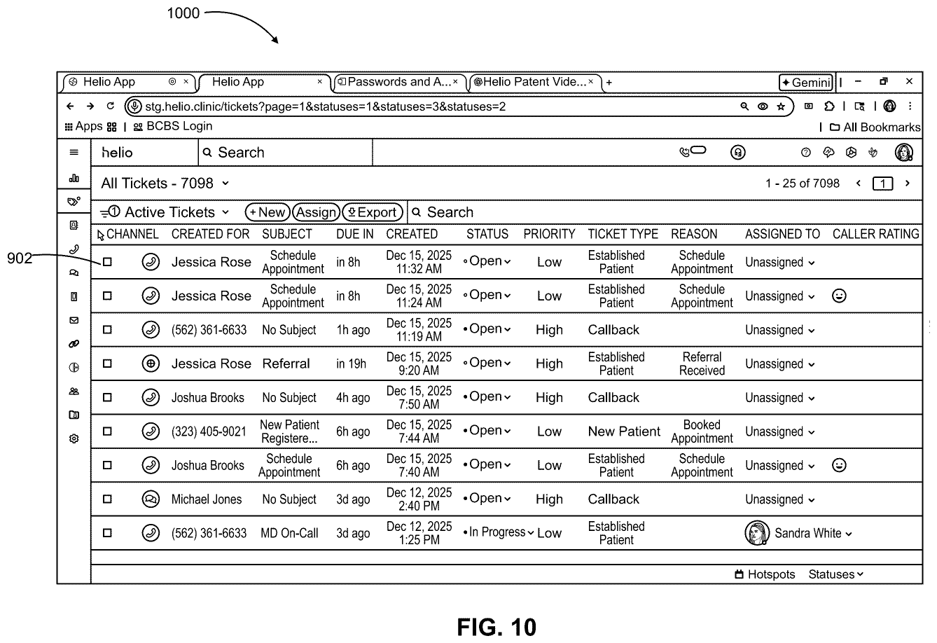

FIG. 10 illustrates an exemplary ticket listing interface 1000. In this embodiment, the interface displays a plurality of tickets 902 including associated contact identifiers, subject matter, due dates, creation timestamps, status indicators, priority levels, and reasons for ticket creation. The interface further indicates whether a given ticket has been automatically assigned to a system user or team and, if so, identifies the assigned user or team. The listing is dynamically updated as tickets are created, classified, assigned, or modified by the system during concurrent processing of communications across multiple channels.

In the exemplary embodiments illustrated in FIG. 6-10, the displayed user interfaces are generated and dynamically updated by the system in response to automated processing steps performed during multi-channel communication orchestration, including contact identity resolution, condition evaluation, communication classification, task generation, assignment, and routing as described with respect to FIGS. 3A-B. As communications are received and processed concurrently across multiple channels, corresponding interface elements are automatically updated to reflect newly created or modified contacts, tickets, assignments, status changes, and activity records, thereby providing real-time visual representations of system state without requiring manual data entry or refresh operations by human users.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents.

### Conclusions, Ramifications, and Scope

While certain embodiments of the invention have been illustrated and described, various modifications are contemplated and can be made without departing from the spirit and scope of the invention. For example, the particular form and content of any user interface associated with certain embodiments of the invention may vary based on the role of each user and might even be capable of personalization according to the needs and/or interests of each individual user. Indeed, it is possible that the user interface may appear different across all types of electronic devices comprising the system as a result of differences in size, operating system, and other specifications defining each electronic device. Accordingly, it is intended that the invention not be limited, except as by the appended claim(s).

The teachings disclosed herein may be applied to other systems, and may not necessarily be limited to any of those particularly described herein. The elements and acts of the various embodiments described above can be combined to provide further embodiments. All of the above patents and applications and other references, including any that may be listed in accompanying filing papers, are incorporated herein by reference. Aspects of the invention can be modified, if necessary, to employ the systems, functions and concepts of the various references described above to provide yet further embodiments of the invention.

Particular terminology used when describing certain features or aspects of the invention should not be taken to imply that the terminology is being refined herein to be restricted to any specific characteristics, features, or aspects of the system, method, and apparatus for facilitating customer relations and business administration with which that terminology is associated. In general, the terms used in the following claims should not be constructed to limit the system, method, and apparatus for facilitating customer relations and business administration to the specific embodiments disclosed in the specification unless the above description section explicitly defines such terms. Accordingly, the actual scope encompasses not only the disclosed embodiments, but also all equivalent ways of practicing or implementing the disclosed system, method and apparatus. The above description of embodiments of the system, method, and apparatus for facilitating customer relations and business administration is not intended to be exhaustive or limited to the precise form disclosed above or to a particular field of usage.

While specific embodiments of, and examples for, the method, system, and apparatus are described above for illustrative purposes, various equivalent modifications are possible for which those skilled in the relevant art will recognize.

While certain aspects of the method and system disclosed are presented below in particular claim forms, various aspects of the method, system, and apparatus are contemplated in any number of claim forms. Thus, the inventor reserves the right to add additional claims after filing the application to pursue such additional claim forms for other aspects of the system, method, and apparatus for facilitating customer relations and business administration.

What is claimed is:

1. A computer-implemented system, comprising:
a computing device comprising a processor, a memory, a network interface, an input/output interface, and a display, the processor being operatively coupled to the memory and the display, and the computing device being operative to execute computer-readable instructions stored in the memory; and
an application comprising machine readable instructions stored in the memory that, when executed by the processor, cause the computing device to at least:
register a user by securely storing user-specific data within the memory;
assign a role to the user based on predefined parameters stored in the memory;
responsive to securely receiving an individualized set of user-specific credentials via the input/output interface, authenticate the user's identity and role;
responsive to authenticating the user's identity, and according to the role of the user, dynamically generate and display via a graphical user interface rendered on the computing device display based on the assigned role of the user a personalized subset of administration modules selected from communication, billing, service, marketing and analytics, and task modules;
responsive to receiving a selection of any one of the personalized subset of administration modules, generate and display at the computing device display an interactive dashboard associated with the selected administration module; and
restricting access to any administration modules not associated with the role assigned to the authenticated user;
wherein each administration module is implemented as a microservice and configured for independent deployment and scalability.

2. The system of claim 1, further comprising a database comprising a plurality of individualized patient health records.

3. The system of claim 1, wherein the communication module is defined by a plurality of communication channels and configured to at least
facilitate text and audio communication between users;
facilitate text and audio communication between users and clients;
automatically transmit client notifications;
quantify and display communication metrics; and
receive and display feedback qualifying user communication.

4. The system of claim 1, wherein the billing module is configured to at least
generate and transmit invoices;
receive payments on invoices; and
generate and transmit a record of any payment to an electronic medical records database for storage.

5. The system of claim 1, the service module is configured to at least
generate, transmit and receive any client intake forms;
facilitate remote audio and/or visual customer service;
responsive to receiving client intake forms, transmitting such forms to a records database for storage; and
generating and transmitting a record of any remote customer service to an electronic records database for storage.

6. The system of claim 1, wherein the service module is a telehealth module configured to
generate, transmit and receive any patient intake forms;
facilitate remote audio and/or visual medical care;
responsive to receiving patient intake forms, transmit such forms to an electronic medical records database for storage; and
generate and transmit a record of any remote customer service to an electronic medical records database for storage.

7. The system of claim 1, wherein the marketing and analytics module is configured to at least
generate, transmit, receive, and automate marketing campaigns;
quantify recipient engagement with marketing campaigns; and
direct marketing campaigns to relevant demographics.

8. The system of claim 1, wherein the task module is configured to at least automatically generate one or more user tasks;
generate one or more tickets associated with such tasks;
receive one or more user generated tasks; and
automate user workflow.

9. The system of claim 1, wherein, responsive to receiving the set of user-specific credentials, and according to the role of the user, generating and displaying, at least the communication and task modules.

10. A method, comprising:
by a computing device comprising a processor, a memory, a network interface, an input/output interface, and a display, the processor being operatively coupled to the memory and the display, the computing device being operative to execute computer-readable instructions stored in the memory:
registering a user by securely storing user-specific data within the memory;
assigning a role to the user based on predefined parameters stored in the memory;
receiving, via an input/output interface, a set of authenticated user-specific credentials identifying the user and role;
responsive to receiving the set of authenticated user-specific credentials, and according to the role of the user, dynamically generating and displaying, via a graphical user interface rendered on the computing device display and based on the assigned role of the user, a personalized subset of administration modules selected from communication, billing, service, marketing and analytics, and task modules;
responsive to receiving a selection of any one of the personalized subset of administration modules, generating and displaying, at the computing device display, an interactive dashboard associated with the selected administration module;
restricting access to any administration modules not associated with the authenticated role;
wherein each administration module is implemented as a microservice and configured for independent deployment and scalability.

11. The method of claim 10, further comprising accessing, by the computing device, a database comprising a plurality of individualized client records.

12. The method of claim 11, wherein the client records are patient health records.

13. The method of claim 10, further comprising, by the communication module,
facilitating text and audio communication between users;
facilitating text and audio communication between users and clients;
automatically transmitting client notifications;
quantifying and displaying communication metrics; and
receiving and displaying feedback qualifying user communication;
wherein the communication module is defined by a plurality of communications channels.

14. The method of claim 10, further comprising, by the billing module, at least
generating and transmitting invoices;
receiving payments on invoices; and
generating and transmitting a record of any payment to an electronic medical records database for storage.

15. The method of claim 10, wherein the service module is a telehealth module further comprising, by the telehealth module,
generating, transmitting, and receiving any patient intake forms;
facilitating remote audio and/or visual medical care; and
responsive to receiving patient intake forms, transmitting such forms to an electronic medical records database for storage; and
generating and transmitting a record of any remote medical care to an electronic medical records database for storage.

16. The method of claim 10, further comprising, by the marketing and analytics module,
generating, transmitting, receiving, and automating marketing campaigns;
quantifying recipient engagement with marketing campaigns; and
directing marketing campaigns to relevant demographics.

17. The method of claim 10, further comprising, by the task module, automatically generating one or more user tasks; generating one or more tickets associated with such tasks; receiving one or more user generated tasks; and automating user workflow.

18. The method of claim 10, responsive to receiving the set of user-specific credentials, and according to the role of the user, generating and displaying at least the communication and task modules.
